(19) United States
(12) Patent Application Publication
(10) Pub. No.: US 2023/0405352 A1
(43) Pub. Date: Dec. 21, 2023

(54) MULTI-MODAL NEUROMODULATION AND NEURO-IMAGING SYSTEM

(71) Applicant: Medify, Inc., Marina Del Ray, CA (US)

(72) Inventor: Raymond Lloyd Harrington, III, Marina Del Ray, CA (US)

(21) Appl. No.: 18/151,391

(22) Filed: Jan. 6, 2023

Related U.S. Application Data
(63) Continuation-in-part of application No. PCT/US2021/040589, filed on Jul. 6, 2021.
(60) Provisional application No. 63/048,579, filed on Jul. 6, 2020.

(51) Int. Cl. A61N 5/06; A61N 2/00; A61N 2/02

(52) U.S. Cl. CPC A61N 5/0622; A61N 2/006; A61N 2/02; A61N 2005/0647; A61N 2005/0659; A61N 2005/0626; A61N 2005/0652; A61N 2005/0632

(57) ABSTRACT

A photobiomodulation system that can be configured as a head wearable and user operated device. The device can include a head wearing portion for directing near infrared light therapy to the frontal lobes. Use of the device can result in reduced or mitigated symptoms of neurological or psychiatric disorder.

### CROSS-REFERENCE TO RELATED APPLICATIONS

This application is a continuation-in-part of International Application No. PCT/US2021/040589, filed on Jul. 6, 2021, which is incorporated by reference and claims the benefit of U.S. Provisional Application No. 63/048,579, filed Jul. 6, 2020, which is incorporated by reference herein.

### FIELD OF THE INVENTION

The subject matter of the present disclosure relates generally to a wearable multi-modality neuroimaging and neuromodulation treatment system. More particularly, the present disclosure relates to the neuromodulation field of photobiomodulation, pulsed or repetitive electromagnetic field stimulation, and/or the neuroimaging field of functional near-infrared spectroscopy.

### BACKGROUND

The global neuromodulation markets are aggressively attacking general health disorders, depression, anxiety, substance abuse, and mood disorders. Further ailments are COVID-19, physical performance issues, erectile problems, gynecologic ailments, inflammation issues, wound healing, stress recovery, and sleep issues. These areas suffer the most from the over consumption of medication and treatments lacking efficacy. Some believe that vast over consumption of medication is failing at an alarming rate and the side effects are at an all-time high. And many of not a majority of patients are seeking non-medication alternatives to treat these debilitating ailments. However, non-drug treatments for neurological disorders have very strict prescription criteria, leaving 50-60 million diagnosed patients in the United States alone, for example, desperately seeking alternatives.

Transcranial stimulation with near infrared light has been associated with increased Adenosine Triphosphate biosynthesis and with increased mitochondrial complex IV expression and activity in the prefrontal cortex. Also, increases hippocampal neurogenesis and neuroprotective effect were reported in animal studies. Different pathways relevant for major depressive disorders (“MDD”) are affected by transcranial photobiomodulation. Modern pathophysiological models for MDD associate this disorder with brain hypometabolism, which is characterized by decreased brain glucose consumption and a common feature of many neuro-degenerative diseases.

Studies have shown that near infrared energy can modulate cytochrome C oxidase and therefore stimulate the mitochondrial respiratory chain leading to increased ATP production. Wang et al. “Interplay between up-regulation of cytochrome-c-oxidase and hemoglobin oxygenation induced by near-infrared laser.” Sci Rep 6, 30540 (2016).

Recently there have been several photomodulation devices released onto the market for professional and private use. However, it is not clear that such photomodulation devices are configured to provide optimal or beneficial EMF therapy and/or are designed for mitigating risks of harmful EMF damage generated by such devices. Electromagnetic radiation (EMF) is a process by which energy particles or waves travel though space, but can be beneficial when purposely directed to portions of the body for the treatment of neurological disorders and regulated in power, for example, whenused up to 1.5 Tesla. Radiant Energy is made up of small packets of particles, called photons, with both working synergistically on the biophysical and biochemical pathways of neurons. Photons can travel alone or move around together in synchrony.

The International Agency for Research on Cancer of the World Health Organization considers harmful EMF to be possibly carcinogenic for humans. IARC Press Release N° 208, 31 May 2011. Some research has indicated that low energy electromagnetic frequency, or harmful EMF radiation emissions, can actually affect our neurological cell health, cell communication, body performance, and the levels of hormones and neurotransmitters in human brains.

Exposure to harmful EMF can create excessive free radicals in human tissue, which can be responsible for oxidative damage and DNA fragmentation. Harmful EMFs can also over-activate the sensitive VGCCs (Voltage-Gated Calcium Channels) in cells that help the release of neurotransmitters and hormones. This increase of Calcium ions in the cell can also induce an oxidative stress response. Because the brain has such a high metabolic rate, ROS and Oxidative damage can happen more easily than in other organs. Oxidative damage in the brain is associated with nervous system impairment, which is a disruption in brain hormones and neurotransmitters.

Hormones and neurotransmitters are responsible for many bodily functions, and harmful EMF radiation as a disruptor has been linked to headaches, depression, anxiety, stress and mood, mental activity and memory issues, and many long term mental and neurodegenerative illnesses due to chronic oxidative stress and hormonal shifts. Such illnesses include ADHD, mood disorders, PTSD, autoimmune diseases, neurodegenerative diseases, and neuropsychiatric diseases such as Schizophrenia and major depressive disorder, as well as anxiety and anxiety-related behaviors. These affect have been shown to impact children and adolescents more, as they majority are spending a larger proportion of their lives connected to technology, and their bodies are still developing, making them vulnerable to environmental toxins like harmful EMF.

Scientists and researchers theorize that while non-ionizing radiation is not yet known to break chemical bonds, such radiation can still induce oxidative stress in a cell. Harmful EMF generates Reactive Oxygen Species (ROS) that can lead to excessive free radicals, a common cause of cancer and neuronal damage. In addition, the amount of electrical force EMFs put on the highly-sensitive voltage-gated calcium channels (VGCC) in the cell can cause a vast increase of calcium ions. An increase in intracellular calcium has been linked with many illnesses, specifically Autism. Calcium ions can react to create even more free radicals, also generated by ROS. This imbalance can break down the cell membrane to allow EMF to interact with and/or break DNA strands, potentially causing cell mutations. If a mutated cell does not die and starts to duplicate, cancerous tumors can form. Research has indicated that there may be an increased risk for leukemia in children, but since only a small number of children got this rare condition, researchers concluded that the results could be due to other factors despite the results being repeatable.

The WHO/International Agency for Research on Cancer (IARC) has classified radio frequency electromagnetic fields as possibly carcinogenic to humans (Group 2B—the same grouping as lead and chloroform) based on an increased risk of glioma, a malignant brain cancer, associated with wireless phone use. In one of the most significant studies to date, conducted over 10 years by the National Toxicology Program (NTP), researchers found that rats exposed to RF radiation had significantly higher rates of glioma, as well as malignant schwannoma (a very rare heart tumor), than unexposed rats. The NTP concluded there is clear evidence that male rats exposed to high levels of radio frequency radiation (RFR) like that used in 2G and 3G cell phones developed cancerous heart tumors. There was also some evidence of tumors in the brain and adrenal gland of exposed male rats. NTP Web site, https://ntp.niehs.nih.gov/go/cellphone Nov. 1, 2018. Scientists at the Ramazzini Institute in Italy found similar significant results when they tested 2,448 rats with radio frequency radiation over their lifetimes, and concluded that the RI findings on far field exposure to RFR are consistent with and reinforce the results of the NTP study on near field exposure. Falcioni et al., “Report of final results regarding brain and heart tumors in Sprague-Dawley rats exposed from prenatal life until natural death to mobile phone radiofrequency field representative of a 1.8 GHz GSM base station environmental emission.” Environ Res. 2018 August; 165:496-503. doi: 10.1016/j.envres. 2018.01.037. Epub 2018 Mar. 7. PMID: 29530389.

While there is much debate as to whether harmful EMF radiation is a cause for cancer in humans, one researcher concluded that of the studies that show a biological or health effect from wireless radiofrequency radiation, 14% are industry funded while 86% are independently funded, while studies that showed no effect, 49% are industry funded while 51% are independently funded. Accordingly, the researcher believed that the differences between the industry funded studies and the independently funded stack suggests bias. Jeffry Fawcett, PhD, “WiFi Blues” Arizona Center for Advanced Medicine, https://www.arizonaadvancedmedicine.com/articles/2013/june/wifi-blues-by-jeffry-fawcett-phd/Jun. 26, 2013. Because of the disagreement between researchers, many EMF generating devices that may or may not be safe with regards to potential for causing human tissue damage, are regardless deemed safe.

The potential for tissue damage notwithstanding, there is little debate that harmful EMFs can interfere with the operation of life-critical, implanted medical devices, such as insulin pumps, defibrillators, and pace-makers. Very recently, Apple Computers released EMF warnings related to many of their current, most-popular products. Specifically, it was warned that under certain conditions, magnets and electromagnetic fields of certain devices (e.g., certain Apple wireless headphones, tablets, smart phones, smart watches, etc.) might interfere with medical devices. As an example, Apple warned that implanted pacemakers and defibrillators might contain sensors that respond to magnets and radios when in close contact. It was further recommended by Apple to keep such products a safe distance away from medical devices (more than 6 inches/15 cm apart or more than 12 inches/30 cm apart if wirelessly charging). Apple Computers Support Website, https://support.apple.com/en-us/HT211900 Published Date: Jun. 25, 2021.

While Apple should be lauded for being upfront with the public regarding safety risks and their devices, it is perhaps more discomforting that many of Apple's many competitors remain silent on the matter. It is reasonable to believe that Apple devices are not the only ones capable of interfering with life-critical medical devices, or put another way—it is reasonable to assume that a majority of similar electronic devices can, under some conditions, produce dangerous levels of harmful EMF interference, considering that competing products share similar types of electronic components, communication protocols, and power profiles, and also the same basic function.

### SUMMARY OF THE INVENTION

Embodiments of the invention relate to photobiomodulation devices, systems, and methods of use as summarized in the following paragraphs.

Some embodiments can relate to a transcranial head and/or a systemic wrist photobiomodulation systems. In some embodiments advanced photobiomodulation, advanced deep photobiomodulation, advanced photonics, or advanced deep photonic therapy can be provided using an array of photonic wavelengths from 250 nm to 1150 nm. In some embodiments, transcranial head and systemic wrist devices can work in tandem as a comprehensive system, or independently. In some embodiments, photobiomodulation systems can deliver continuous wave (CW) or pulsed photobiomodulation therapy from lhz to 200 hz.

In some embodiments, systems can include: specialized flexible and/or rigid housing power buttons, and/or a toggle wheel, and/or wifi capability (e.g. Bluetooth), and/or flexible or rigid touchscreen, and/or flexible or rigid circuit boards, and/or flexible or rigid batteries, and/or flexible or rigid (Light emitting diode) LEDs, and/or flexible and/or rigid floating LED holders (that can include modulated cooling therapy or heat sinking foam and/or pressure impact foam), and/or internal or external air cooling capability, and/or rigid or flexible internal or external thermoelectric cooling (TEC) devices applied to the wrists radial artery or forehead (fp1,fp2,fpz,f3,f4 EEG Locations), and/or related software stored in a non-volatile storage medium.

In some embodiments, systems can be configured to determine patient's SP02 rate, pulse rate, skin temperature on wrist and forehead, internal or external device temperature, and other variations known in the art.

In some embodiments, data can be transmitted directly to other devices (e.g., smart phones, tablets, personal computers, servers). In some embodiments, data can be wirelessly transmitted from the system via one or more of local wireless networks, The Internet, and cellular data networks.

In some embodiments, photobiomodulation systems can include modular attachments for focal use applications like thyroid treatment and connections or integration like earlier stated thermoelectric cooling (TEC) directly to cool the forehead. In some embodiments, multiple LEDs of a system can operate in unison for unilateral treatment (Right or Left Pre-Frontal Cortex), bilateral treatment (Right and Left Pre-Frontal Cortex), or independently of each other (e.g., individual LEDs can be powered off or modulated to reduce output while other LEDs are powered on). In some embodiments, one or more LEDs can be operated to apply photobiomodulation therapy in conjunction or at different power levels and frequencies with one or more other LEDs (e.g., one or more LEDs applying a certain power or frequency therapy to the left brain hemisphere and one or more LEDs applying a different power level or frequency therapy to the right brain hemisphere).

In some embodiments, systems can include total photon capture technology (TPC Technology). In some embodiments, TPC optical devices can range in size from 2 mm-100 mm. In some embodiments, TPC optical devices can be lenses and can have circular, oval, ellipse, square, rectangle, rhombus, pentagon, hexagon, heptagon, octagon, nonagon, and decagon shapes. In some embodiments, TPC lenses can disburse in ultra narrow, narrow, tight spot, medium spot, wide spot, and diffused spot. In some embodiments, lenses can be configured for total internal reflection or standard reflection. In some embodiments, TPC lenses can have a full width at half maximum diffusion value ranging from 2 to 90 degrees. In some embodiments, TPC lenses can have a viewing angle that can range from 1 degree to 130 degrees. In some embodiments, TPC lenses can have a mechanical lens holding portions configured for/as direct connection, lens holding, lenses with legs, and lenses with rim. In some embodiments, mechanical lens holding portions can hold between 1-6 LEDs per lens (multi lens component). In some embodiments, TPC lenses can be placed in component foam, glued in place, double taped, double tape cushion, or manufactured together. In some embodiments, TPC optical devices can be single lens or arranged as an infinite rigid or flexible configuration embodying the entire LED configuration. In some embodiments, a TPC optical device panel can include a flexible arrangement of lenses and LEDs fused together to embody all LED's encompassed in photobiomodulation device.

Some embodiments are related to a photobiomodulation system that can include a housing that can be shaped to fit against the outer surface of a human head about the frontal lobe. A plurality of LED emitters can be carried by the housing. The plurality of LED emitters can be configured to provide therapeutic near infrared light to the frontal lobe for treating neurological disorders. An operating circuit can operate the LED emitters. In some embodiments, the operating circuit can emit or power a beneficial electromagnetic field (“EMF”) stimulation plates or coils to FP1, FP2, Fz, F3, F4, F7, F8, e.g., from 0.01 to 1.5 Tesla during operation of the LED emitters. In some embodiments, the housing can also be configured to shield, deflect, or emit pulsed/repetitive electromagnetic stimulation, e.g., of 0.01 to 1.5 Tesla to a patient wearing the housing which can effectively counter harmful EMF radiation and provide therapeutic benefits.

In some embodiments, a pulsed/repetitive electromagnetic stimulator or an EMF shield deflector can be positioned to redirect the beneficial EMF radiation from the harmful EMF during operation of the LED emitters. In some embodiments, some embodiments can position single or multiple electromagnetic coil(s), plate(s), or sheet(s) to EEG sites of Fz, Fp1, Fp2, F3, F4, Fz, F7, or F8 of a patients targeted brain area. In some embodiments the electromagnetic field of stimulation can further target a single area as in EEG site F3, or a variation of EEG sites as in F3 and F4 simultaneously. In some embodiments, single or multiple pulsed or repetitive electromagnetic stimulating coils can be configured to provide transcranial magnetic stimulation.

In some embodiments, an EMF shield can be positioned to redirect the beneficial EMF radiation from the harmful EMF during operation of the LED emitters.

In some embodiments, the housing can be curved or customized to match the shape of each human forehead region and the housing can include an inner housing portion for holding at least a portion or all of the LED emitters.

In some embodiments, the housing can include an outer housing portion or tethered housing portion for holding the operating circuit.

In some embodiments, the beneficial EMF emitting shield can be positioned directly at FP1, FP2, Fz, F3, F4, F7, F8 from the inner/outer housing portion and inner/outer housing portion.

In some embodiments, the beneficial EMF shield can separate light emitting portions of the LED emitters from the operating circuit.

In some embodiments, the beneficial EMF shield can be a metal foil, coil plate, mesh, a metalized coil layer on a surface of the housing a substrate, or a metal/polymer composite material.

In some embodiments, the housing can be formed from an beneficial EMF metal or composite material configured to shield the patient wearing the housing from the harmful EMF radiation.

In some embodiments, the LED emitters can be configured to emit near infrared light at a wavelengths between 800 and 810 nm. 650 to 900 nanometers, and 650 to 1150 nanometers.

In some embodiments, the LED emitters can be configured to emit near infrared light at a power density of 25 mW/cm2, 50 mW/cm2, 75 mW/cm2, 100 mW/cm2, 125 mW/cm2, 150 mW/cm2, 175 mW/cm2, 200 mW/cm2, 225 mW/cm2, 275 mW/cm2, 300 mW/cm2, 235 mW/cm2

In some embodiments, the LED emitters can be configured to emit near infrared light at a power density of 25 to 325 mW/cm2.

In some embodiments, the LED emitters can be configured to emit the near infrared light at frontal lobe locations corresponding to FP1, FP2, Fz, F3, F4, F7, F8 EEG positions.

In some embodiments, the LED emitters can be configured to emit the near infrared light only at frontal lobe locations corresponding to FP1, FP2, F7, F8 EEG positions.

In some embodiments, the operating circuit can include a wireless communication device, and wherein the wireless communication device causes at least most of the harmful EMF radiation emitted during operation of the LED emitter.

In some embodiments, the housing can be configured to shield the patient wearing the housing from 95-99.9% of the harmful EMF radiation

### BRIEF DESCRIPTION OF THE DRAWINGS

For a better understanding of at least certain embodiments, reference will be made to the following Detailed Description, which is to be read in conjunction with the accompanying drawings.

FIGS. 1A-1C are perspective views of a photobiomodulation therapy device, according to some embodiments.

FIG. 2A is a schematic view of a photobiomodulation therapy device, according to some embodiments.

FIG. 2B is an exploded view of a portion of a photobiomodulation therapy device, according to some embodiments.

FIGS. 3 and 4 are perspective views of a photobiomodulation therapy device, according to some embodiments.

FIGS. 5, 6, and 7 are schematic drawings of light emitters, according to some embodiments.

FIGS. 8 and 9 are schematic drawings of LED housings, according to some embodiments.

FIGS. 10A and 10B are perspective views of a near infrared spectroscopy imaging system, according to some embodiments.

FIG. 11 is a schematic of a computing system, according to some embodiments.

The figures depict various embodiments of the present invention for purposes of illustration only, wherein the figures use like reference numerals to identify like elements. One skilled in the art will readily recognize from the following discussion that alternative embodiments of the structures and methods illustrated in the figures may be employed without departing from the principles of the invention described herein.

### DETAILED DESCRIPTION

Embodiments are disclosed that relate to devices for providing photobiomodulation therapy.

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation. The verb “can” is used herein to identify optional elements, when not otherwise explicitly identified as “optional” and is also defined to mean “can or cannot” (the latter of which is not repeated throughout this disclosure for the sake of brevity and readability).

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order that is logically possible.

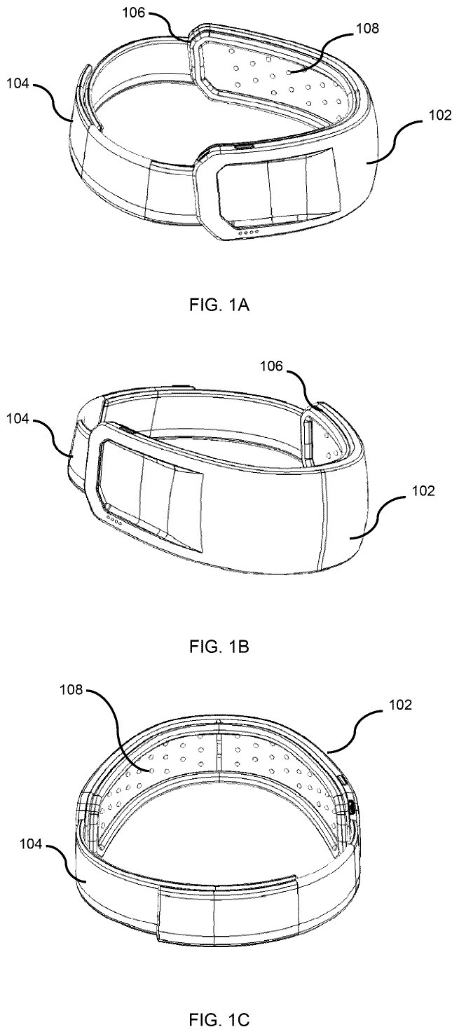

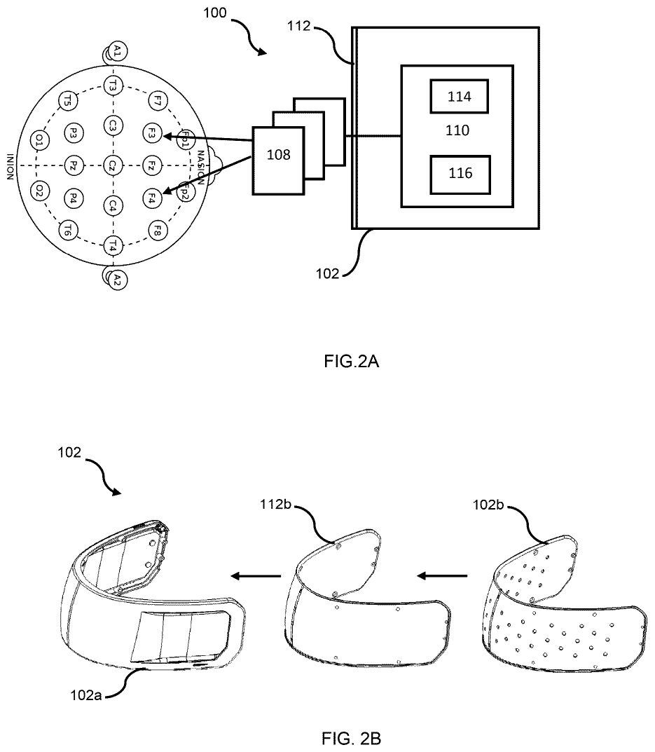

FIGS. 1A-1C show various view of photobiomodulation device 100, according to some embodiments. Device 100 can include a housing 102, which can be shaped to conform to a forehead region of a human head. In some embodiments, housing 102 is shaped to supply photobiomodulation therapy to particular regions of a patient's head, such as regions corresponding to the International 10-20 EEG placement system for applying scalp electrodes for an EEG exam. Such regions, for example, can include the F2, F3, F4, F7, F8, Fp1, and/or Fp2 locations per the 10-20 EEG system. However, for purposes of this disclosure, in some embodiments, photobiomodulation need not be applied solely to such locations, rather, in some embodiments such locations can act as general zones for application of photobiomodulation therapy, for example, photobiomodulation therapy can be applied between and/or across any one, two, or more of the F2, F3, F4, F7, F8, Fp1, and/or Fp2 EEG locations (see head H at FIG. 2A). In some embodiments, photobiomodulation therapy can be applied at the FP1, FP2, Fz, F3, F4, F7, F8 locations. In some embodiments, photobiomodulation therapy is only applied at the FP1, FP2, Fz, F3, F4, F7, F8 locations and nowhere else. The patient may not be able to always accurately place device 100 on their head in a precisely correct position (i.e., so that particular light emitters centers perfectly over the EEG locations) and for the sake of practicality device 100 may be of only one or two possible sizes (i.e., different sized patients will have different sized heads), thus some over-coverage of the locations can be helpful less sensitivity to mitigate placement. Accordingly, housing 102 can have a curved shape for placement directly adjacent to the frontal lobe for covering one or more of the EEG locations.

In an open trial with a single session of near infrared PBM for treatment resistant depression on the forehead on EEG sites F3/F4, which cover the dorsolateral prefrontal cortex (DLPFC) bilaterally showed significant decrease in depression symptoms at 2 weeks and 4 weeks post treatment. Schiffer et al. “Psychological benefits 2 and 4 weeks after a single treatment with near infrared light to the forehead: a pilot study of 10 patients with major depression and anxiety.” Behavioral and brain functions: BBF vol. 5 46. 8 Dec. 2009, doi:10.1186/1744-9081-5-46. In another study that included two sessions per week for a total of three weeks of bilateral forehead near infrared (808 nm) treatment, showed decreases in depression symptoms. Cassano et al. “Near-Infrared Transcranial Radiation for Major Depressive Disorder: Proof of Concept Study.” Psychiatry J. 2015; 2015:352979. doi:10.1155/2015/352979

A randomized control trial to assess the efficacy of transcranial PBM as primary treatment for Major Depressive Disorder. Bilateral treatment at EEG F3 and F4 sites twice a week for 8 weeks versus a sham treatment, showed greater depression remission rates compared to the sham. Cassano et al. “Transcranial Photobiomodulation for the Treatment of Major Depressive Disorder. The ELATED-2 Pilot Trial.” Photomed Laser Surg. 2018 Dec; 36(12):634-646. doi: 10.1089/pho. 2018.4490. Epub 2018 Oct. 20. PMID: 30346890; PMCID: PMC7864111.

In some embodiments, housing 102 can include adjustable head strap 104, which can for example, be constructed from flexible and stretchable fabric and include a hook/loop fastening mechanism and be attached to ends of housing 102. In some embodiments, housing 102 can be constructed from many different rigid materials, such as polymers, metal alloys, and composite (e.g., carbon fiber, fiberglass) materials. In other embodiments, housing 102 can be constructed from non-rigid and elastic materials, such as a resilient or malleable low durometer (i.e., rubber-like) polymers or fabric materials. In some embodiments, housing 102 can include a foam or rubber/silicon sealing forhead gasket 106 that deforms to conform to the shape of a patient's frontal lobe region. In use, a patient can apply sealing gasket 106 and connected housing 102 to their forehead region and stretch/adjust/tighten headstrap 104 to maintain device 100 in place about the frontal lobes.

An inner facing portion of housing 102 includes an array of emitters 108 for providing one or more types of photobiomodulation therapy. Emitters are specified according to the type of photobiomodulation therapy provided and can be light emitting diodes (LED), laser, or broadband lights for example. In some embodiments, emitters 108 can produce light of a single wavelength, such as a wavelength in the near-infrared or infrared spectrum. In some embodiments, emitters 108 can be non-uniformly configured so as to include emitters configured to produce light of different wavelengths, such as a first, second, third, and/or fourth wavelength in the near-infrared or infrared spectrum.

In some embodiments, emitters 108 can produce light having a wavelength greater or equal to 650 nm and less than or equal to 1150 nm. In some embodiments, emitters 108 can produce light having a wavelength greater or equal to 750 nm and less than or equal to 850 nm, greater or equal to 785 nm and less than or equal to 835 nm, or greater or equal to 800 nm and less than or equal to 1150 nm. Some studies have found that light between 800 and 810 nm is ideal for deep penetration into frontal lobe brain tissue, including at the FP1, FP2, Fz, F3, F4, F7, F8 EEG locations. In some embodiments, emitters 108 can be configured to output a power density greater or equal to 25 mW/cm² and less than or equal to 325 mW/cm², such as 25 mW/cm², 50 mW/cm², 75 mW/cm², 100 mW/cm², or 125 mW/cm², 250 mW/cm², 325 mW/cm². In some embodiments, emitters 108 can be configured to continuously output light over time and/or pulse light over time. In some embodiments, emitters 108 can be configured to pulse light at a frequency within a range of 1-200 MHz. In some embodiments, emitters 108 can be configured to pulse light at a frequency of 200 MHz.

FIG. 2A shows a simplified schematic layout of device 100, according to some embodiments. Housing 102 can hold electronic circuit 110, which can operate device 100. Circuit 100 can include various aspects such as processors, memory, batteries, LED drivers, and wireless communication devices, which are discussed in more detail below. Circuit 100 is separated from emitters 108 by shield 112, which is configured to block harmful electromagnetic radiation (EMF) emitted by circuit 110. In some embodiments, circuit 110 can include a power circuit 114, which can include a rechargeable battery and/or line power source/DC conversion circuit. In some embodiments, circuit 110 can include a communication circuit 116, which can be a wireless or wired circuit configured to transmit instructions and/or data to and from circuit 110.

In some embodiments, shield 112 can help mitigate any potential interference caused by circuit 100 and the patient using device 100. In some embodiments, shield 112 can be configured to block 95 to 99.9% of harmful wireless EMF and 5G RF radiation (300 Hz-10 GHz). In some embodiments, shield 112 can be configured to conduct, absorb, and dissipate up to 99.9% of EMF radiation from 0-10 GHz. In some embodiments, shield 112 can be configured to block 95-99.9% of EMF radiation, which includes 5G, cellular, WiFi, and Bluetooth. In some embodiments, shield 112 can be configured to block 95-99.9% of Extremely Low Frequency (ELF) emissions. In some embodiments, shield 112 can attenuate more 30-40 dB, 40-50 dB, 60-90 dB, or 90-120 dB.

In some embodiments, upon a receiving a start command (e.g., enacted by a user pressing a start button coupled to circuit 110), circuit 110 can be configured (e.g., by processor executable computer instructions stored on non-volatile, tangible computer-readable memory) to operate emitters 108 for the calculated treatment duration to generate near infrared light a wave length of 650 to 1150 nm and power density of 25 to 325 mW/cm². In some embodiments, the light is directed at the at the FP1, FP2, Fz, F3, F4, F7, F8 EEG locations of a patient wearing device 100 to only deliver the near infrared light to the frontal lobes at the FP1, FP2, F3 and F4 EEG locations and in some embodiments only to the frontal lobes at the F3 and F4 EEG locations. Hence, in some embodiments, additional emitters 108 can exist and be positioned in relation to non-F1, non-F2, non-F3 and non-F4 EEG locations are intentionally not operated according to the processor executable computer instructions. In some embodiments, during this process, circuit 110 generates potentially harmful EMF radiation and 95-99.9% of the harmful EMF radiation that would otherwise be directed to the patient, is countered, blocked, redirected by beneficial EMF shield 112. In some embodiments, this beneficial EMF process can reduce or mitigate symptoms of depression.

Shield 112 can be constructed from a metal, such as nearly pure or alloyed metal. In some embodiments, shield 112 can be a metal coil plate, mesh, one or more deposited/plated layer on a substrate, metal/polymer composite material. In some embodiments, shield 112 is integrated within housing 102 to such extent that they are the same component, i.e., a shield-housing, for example, a metal housing, a metal coated housing, a metal fabric housing, a metal composite material, or integrated iron coils. In some embodiments, shield 112 can be configured to generate a therapeutic energy configured to shield, deflect, or emit pulsed/repetitive electromagnetic stimulation of 0.01 to 1.5 Tesla to a patient wearing the housing which can effectively counter harmful EMF radiation and provide therapeutic benefits. For design considerations see: Somani A, Kar S K. Efficacy of repetitive transcranial magnetic stimulation in treatment-resistant depression: the evidence thus far. Gen Psychiatr. 2019. In some embodiments, shield 112 can position single or multiple electromagnetic coil(s), plate(s), or sheet(s) to EEG sites of Fz, Fp1, Fp2, F3, F4, Fz, F7, or F8 of a patients targeted brain area. The electromagnetic field of stimulation can further target a single area as in EEG site F3, or a variation of EEG sites as in F3 and F4 simultaneously. In some embodiments, the housing can also be configured to shield, deflect, or emit pulsed/repetitive electromagnetic stimulation, e.g., of 0.01 to 1.5 Tesla to a patient wearing the housing which can effectively counter harmful EMF radiation and provide therapeutic benefits. For design considerations see: Deng Z D, Lisanby S H, Peterchev A V. Coil design considerations for deep transcranial magnetic stimulation. Clin Neurophysiol. 2014 Jun; 125(6):1202-12. doi: 10.1016/j.clinph. 2013.11.038. Epub 2013 Dec. 22. PMID: 24411523; PMCID: PMC4020988., and Mengfei Liu, et al., “Electromagnetic and mechanical characterization of a flexible coil for transcranial magnetic stimulation”, AIP Advances 9, 035335 (2019) https://doi.org/10.1063/1.5080148.

Generally, many different types of metal can be used for shield 112, with perhaps solid copper sheet having the best shielding capabilities when viewed in a vacuum, while iron coils can have the best treatment field parameters. Selection of a shield or electromagnetic field metal material is the basis of the level of shielding (attenuation), deflecting, or electromagnetic field output required for clinical magnetic stimulation output of device 100. Such output to achieve 0.01 to 1.5 Tesla can vary based on the power storage, power consumption, quality of shielding intracomponent connections to prevent EMF bleeding, and communication power output.

In some embodiments, upon a receiving a start command (e.g., enacted by a user pressing a start button coupled to circuit 110), circuit 110 can be configured (e.g., by processor executable computer instructions stored on non-volatile, tangible computer-readable memory) to operate emitters 108 for treatment duration to generate near infrared light a wave length of 650 to 1150 nm and power density of 25 to 325 mW/cm². In some embodiments, the light is only directed at the at the F3 and F4 EEG locations of a patient wearing device 100 to deliver the near infrared light to the frontal lobes at the F3 and F4 EEG locations and in some embodiments only to the frontal lobes at the F3 and F4 EEG locations. Hence, in some embodiments, additional emitters 108 can exist and be positioned in relation to non-F3 and non-F4 EEG locations are intentionally not operated according to the processor executable computer instructions. In some embodiments, during this process, circuit 110 generates EMF radiation and 95-99.9% of the EMF radiation that would otherwise be directed to the patient, is blocked by shield 112. In some embodiments, this process can reduce or mitigate symptoms of depression. In some embodiments,

FIG. 2B shows an exploded view of housing 102, according to some embodiments. Housing 102 includes outer housing 102a and inner housing 102b. In some embodiments, circuit 110 can be mounted or integrated into one or more of outer housing 102a and inner housing 102b. Emitters 108, or a light emitting portion thereof, can be mounted to inner housing 102b. Shield 112b separates outer housing 102a and light emitting portions of emitters 108 with circuit 110 (and in some embodiments driving circuits for emitters 108, where said driving circuits can be physically separated from or integrated with circuit 110). Shield 112b can be a metal foil, plate, mesh, one or more deposited/plated layer on inner housing 102b and in some embodiments on outer housing 102a. In some embodiments, shield 112b is not physically separated from inner housing 102b and outer housing 102a, and accordingly some or all of inner housing 102b and outer housing 102a can be formed from a composite shielding material or metal material.

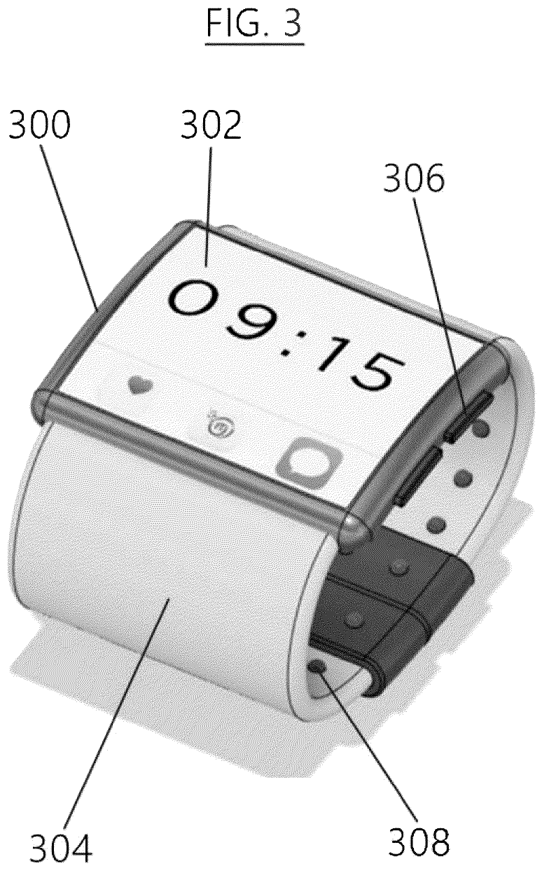

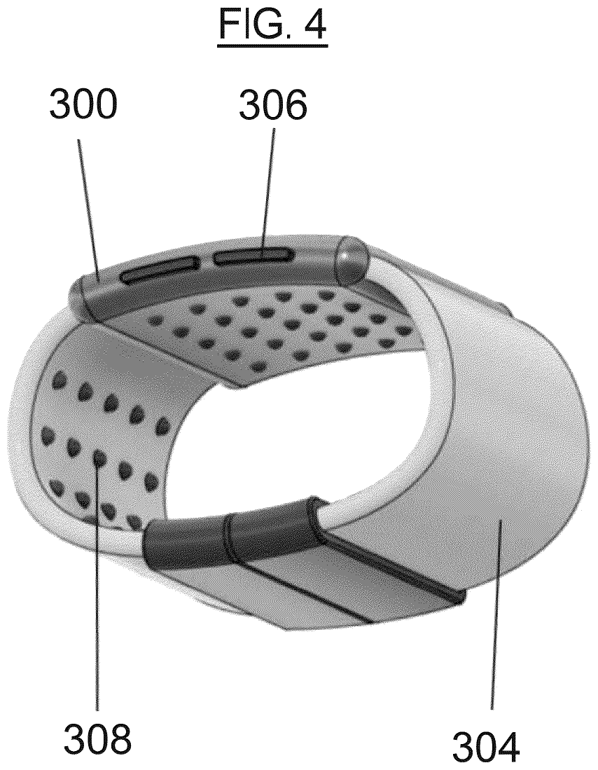

FIGS. 3 and 4 are illustrative representations of a wearable systemic radial artery wrist photobiomodulation system, in top/side view, according to some embodiments. Wearable systemic radial artery wrist photobiomodulation system 300, can take the form of a smartwatch or standard wristband with touchscreen 302 and wrist strap 304. As stated earlier, the systemic photobiomodulation wristband can include a rigid or flexible thermoelectric cooling plate encompassing the wrist or forearm including radial artery. This rigid or flexible plate is designed to touch against the skin and provide direct cooling. Two buttons on the side of the device provide additional inputs 306. Photobiomodulation therapy is delivered through a series of emitters 308, which can be configured as described with respect to emitters 108.

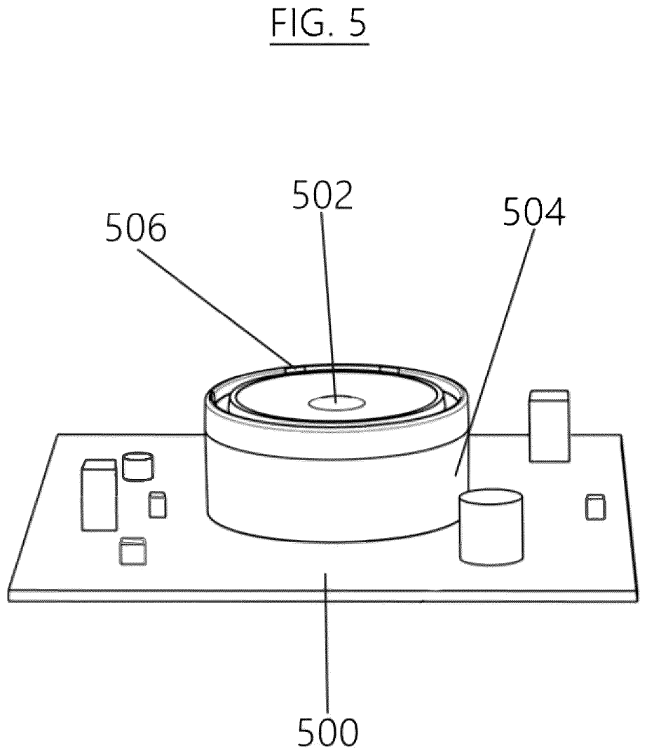

FIG. 5 is an illustrative representation of an LED emitter (which can include emitters 108/308), according to some embodiments. In some embodiments, printed circuit board (“PCB”) 500 can support LED 502 and optical casing 504. Holder 506 enables assembly of assembling the lens easier and can enhance lens performance.

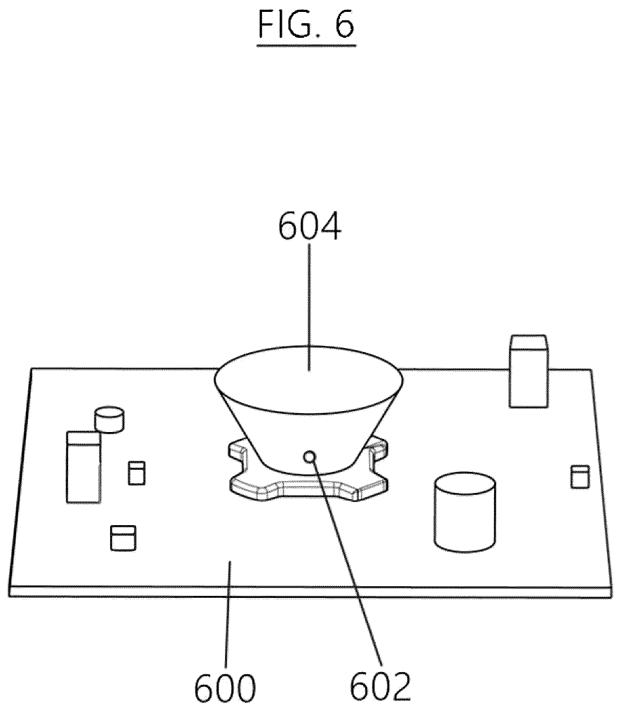

FIG. 6 is an illustrative representation of an LED emitter (which can include emitters 108/308), according to some embodiments. In some embodiments, PCB 600 can support an LED 602 and optical casing 604. LED 602 can be housed within optical casing 604.

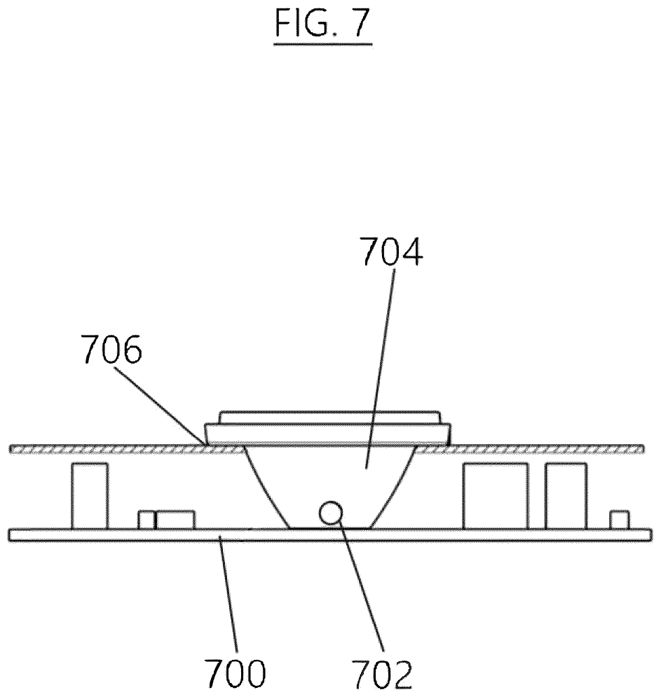

FIG. 7 is an illustrative representation of an LED emitter (which can include emitters 108/308), according to some embodiments. In some embodiments, PCB 700 supports LED 702 and optical casing 704. LED 702 can be housed within optical casing 704. Substrate 706 include shielding aspects of shield 112.

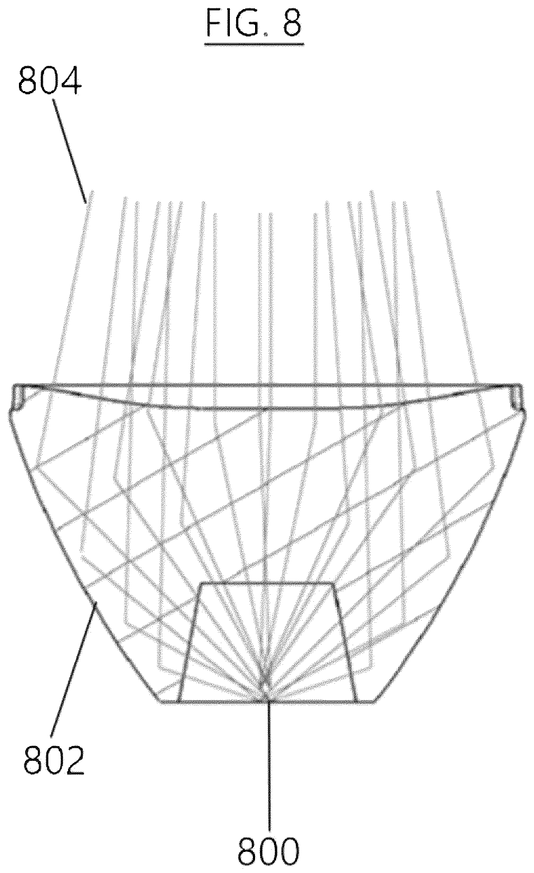

FIG. 8 is an illustrative representation of an LED and total internal reflector casing emitter (which can be incorporated into emitters 108/308), according to some embodiments. LED 800 emits photons from within a Total Photon Capture (TPC Technology) casing 802. This can create highly focused (non-diffuse) photons 804.

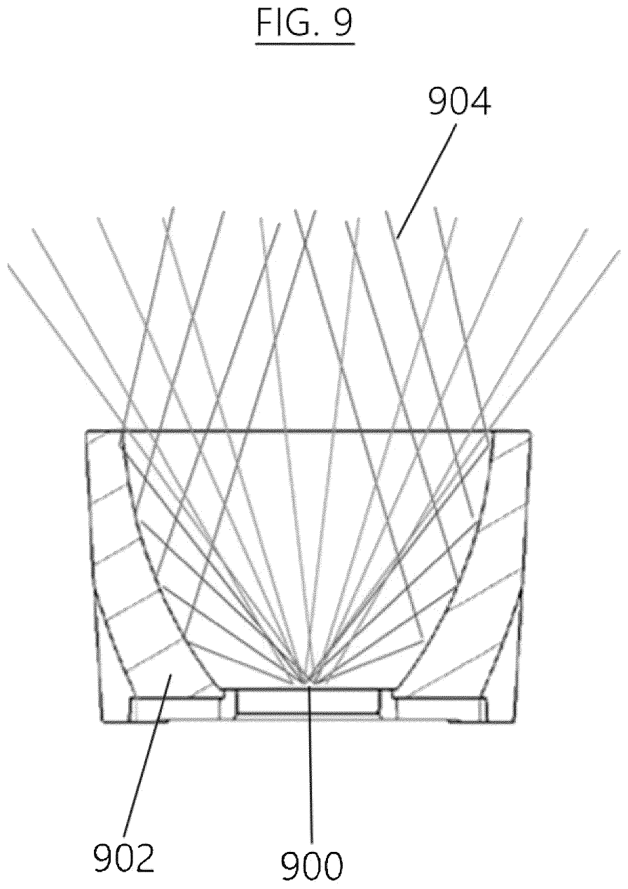

FIG. 9 is an illustrative representation of an LED and reflector casing (which can be incorporated into emitters 108/308), in accordance with some embodiments. LED 900 can emit photons from within a Total Photon Capture (TPC Technology) casing 902. Lines 904 indicate beams that are reflected by the reflector surface.

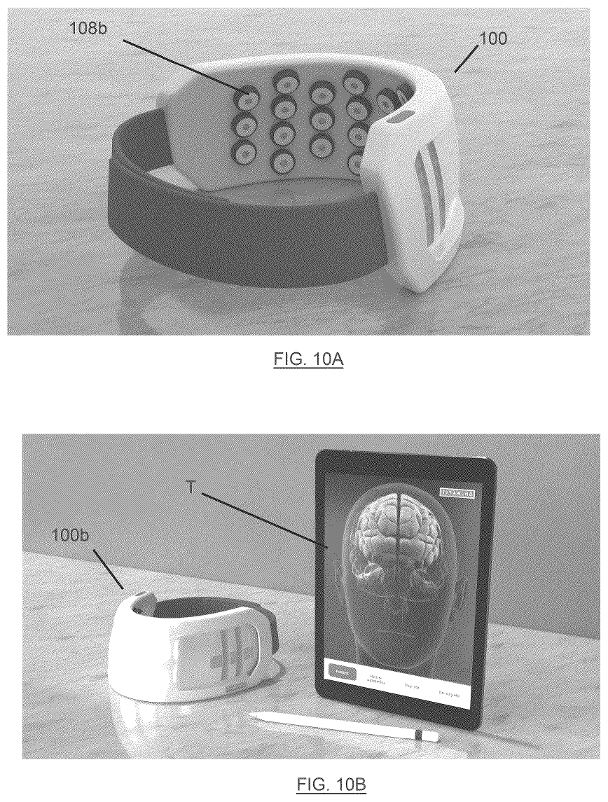

As depicted at FIGS. 10A and 10B, in some embodiments, device 100 emitters 108 can include detection components for a near infrared spectroscopy imaging system. Device 100b can be configured largely in the same manner described with respect to device 100, but with inclusion of emitter/detector 108B. Emitter/detector 108b can be configured as part of an optical detection system that can include an optical lens and an optical detector array. Details of such arrays are disclosed within U.S. Pat. No. 9,545,223, which is incorporated by reference herein. In some embodiments, device 100b can be configured to communicate with portable computing device T to show real time imaging scans.

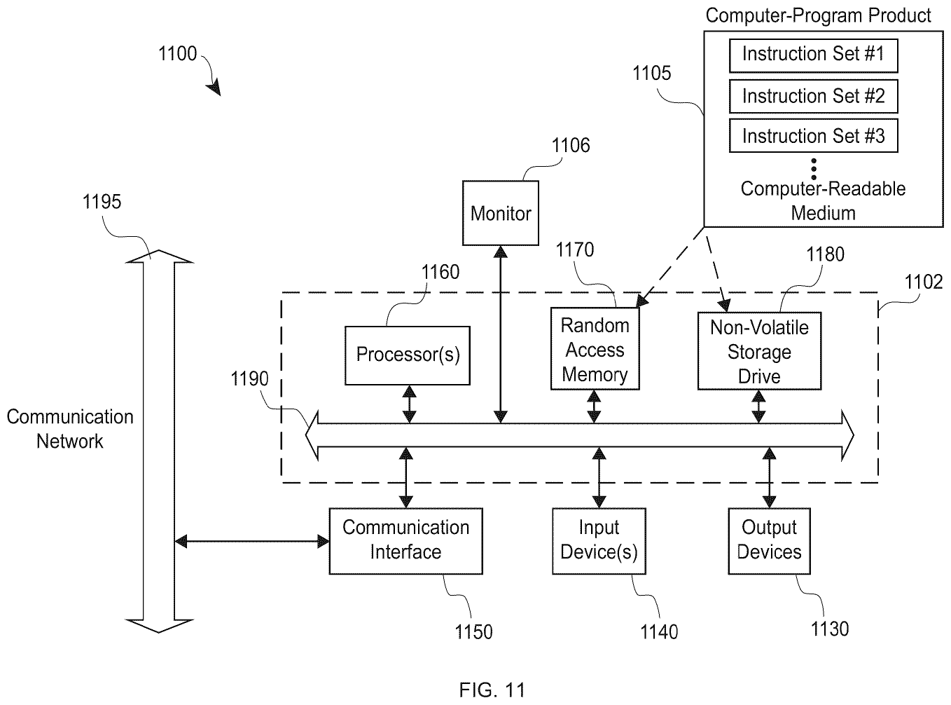

With reference to FIG. 11, an embodiment of a special-purpose computer system 1100 is shown, which in some embodiments can include circuit 110. For example, one or more intelligent components, processing system 110 and components thereof may be a special-purpose computer system 1100. Such a special-purpose computer system 1100 may be incorporated as part of any of the other computerized devices discussed herein, such devices shown at FIGS. 1A-1C, 2A, 2B, and 3-6. Methods disclosed herein may be implemented by computer-program products that direct a computer system to perform the actions of the above-described methods and components. Each such computer-program product can include sets of instructions (codes) embodied on a non-volatile, computer-readable medium that direct the processor of a computer system to perform corresponding actions. The instructions can be configured to run in sequential order, or in parallel (such as under different processing threads), or in a combination thereof. After loading the computer-program products on a general-purpose computer system 1126, it can be transformed into the special-purpose computer system 1100.

Special-purpose computer system 1100 can include a computer 1102, a monitor 1106 coupled to computer 1102, one or more additional user output devices 1130 (optional) coupled to computer 1102, one or more user input devices 1140 (e.g., keyboard, mouse, track ball, touch screen) coupled to computer 1102, an optional communications interface 1150 coupled to computer 1102, a computer-program product 1105 stored in a non-volatile, tangible computer-readable memory in computer 1102. Computer-program product 1105 directs computer system 1100 to perform the above-described methods. Computer 1102 may include one or more processors 1160 that communicate with a number of peripheral devices via a bus subsystem 1190. These peripheral devices may include user output device(s) 1130 (e.g., emitters 108 and driving circuitry), user input device(s) 1140, communications interface 1150, and a storage subsystem, such as random-access memory (RAM) 1170 and non-volatile storage drive 1180 (e.g., disk drive, optical drive, solid state drive), which are forms of tangible computer-readable memory.

Computer-program product 1105 may be stored in non-volatile storage drive 1180 or another computer-readable medium accessible to computer 1102 and loaded into random access memory (RAM) 1170. Each processor 1160 may comprise a microprocessor, such as a microprocessor from Intel® or Advanced Micro Devices, Inc.®, or the like. To support computer-program product 1105, the computer 1102 runs an operating system that handles the communications of computer-program product 1105 with the above-noted components, as well as the communications between the above-noted components in support of the computer-program product 1105. Exemplary operating systems include Windows® or the like from Microsoft Corporation, Solaris® from Sun Microsystems, LINUX, UNIX, and the like.

User input devices 1140 include all possible types of devices and mechanisms to input information to computer 1102. These may include a keyboard, a keypad, a mouse, a scanner, a digital drawing pad, a touch screen incorporated into the display, audio input devices such as voice recognition systems, microphones, and other types of input devices. In various embodiments, user input devices 1140 are typically embodied as a computer mouse, a touch screen, camera, wireless remote, drawing tablet, or voice command system. User input devices 1140 can allow a user to select, input, or add objects, icons, text, photos, and the like that appear on the monitor 1106 via a command such as a click of a button or the like. User output devices 1130 include various types of devices to output information from computer 1102. These may include a display (e.g., monitor 1106), printers, non-visual displays such as audio output devices, etc.

Communications interface 1150 provides an interface to other communication networks, such as communication network 1195, and devices and may serve as an interface to receive data from and transmit data to other systems, WANs and/or the Internet. Embodiments of communications interface 1150 typically include an Ethernet card, a modem (telephone, satellite, cable, ISDN), a (asynchronous) digital subscriber line (DSL) unit, a USB interface, a wireless network adapter, and the like. For example, communications interface 1150 may be coupled to a computer network, or the like. In other embodiments, communications interface 1150 may be physically integrated on the motherboard of computer 1102, and/or may be a software program, or the like.

RAM 1170 and non-volatile storage drive 1180 are examples of tangible computer-readable media configured to store data such as computer-program product embodiments of the present invention, including executable computer code, human-readable code, or the like. Other types of tangible computer-readable media include floppy disks, removable hard disks, optical storage media such as CD-ROMs, DVDs, bar codes, semiconductor memories such as flash memories, read-only-memories (ROMs), battery-backed volatile memories, networked storage devices, and the like. RAM 1170 and non-volatile storage drive 1180 may be configured to store the basic programming and data constructs that provide the functionality of various embodiments of the present invention, as described above.

Software instruction sets that provide the functionality of the present invention may be stored in RAM 1170 and non-volatile storage drive 1180. These instruction sets or code may be executed by the processor(s) 1160. RAM 1170 and non-volatile storage drive 1180 may also provide a repository to store data and data structures used in accordance with the present invention. RAM 1170 and non-volatile storage drive 1180 may include a number of memories including a main random-access memory (RAM) to store instructions and data during program execution and a read-only memory (ROM) in which fixed instructions are stored. RAM 1170 and non-volatile storage drive 1180 may include a file storage subsystem providing persistent (non-volatile) storage of program and/or data files. RAM 1170 and non-volatile storage drive 1180 may also include removable storage systems, such as removable flash memory.

Bus subsystem 1190 provides a mechanism to allow the various components and subsystems of computer 1102 to communicate with each other as intended. Although bus subsystem 1190 is shown schematically as a single bus, alternative embodiments of the bus subsystem may utilize multiple busses or communication paths within the computer 1102.

Throughout the foregoing description, and for the purposes of explanation, numerous specific details are set forth in order to provide a thorough understanding of the described techniques. It will be apparent, however, to one skilled in the art that these techniques can be practiced without some of these specific details. Although various embodiments that incorporate these teachings have been shown and described in detail, those skilled in the art could readily devise many other varied embodiments or mechanisms to incorporate these techniques. Also, embodiments can include various operations as set forth above, fewer operations, or more operations; or operations in an order. Accordingly, the scope and spirit of the invention should be judged in terms of the claims, which follow as well as the legal equivalents thereof.

What is claimed is:

1. A photobiomodulation system comprising:
a housing shaped to fit against the outer surface of a human head about the frontal lobe;
a plurality of LED emitters carried by the housing, the plurality of LED emitters configured to provide therapeutic near infrared light to the frontal lobe for treating neurological disorders;
an operating circuit for operating the LED emitters, wherein the operating circuit emits electromagnetic field (“EMF”) radiation during operation of the LED emitters;
wherein the housing is configured to shield a patient wearing the housing from the EMF radiation.

2. The photobiomodulation system of claim 1, further comprising an EMF shield positioned to block the EMF radiation from the patient during operation of the LED emitters.

3. The photobiomodulation system of claim 2, wherein the housing is curved to match the shape of a human forehead region and wherein the housing includes an inner housing portion for holding at least a portion of the LED emitters.

4. The photobiomodulation system of claim 3, wherein the housing includes an outer housing portion for holding the operating circuit.

5. The photobiomodulation system of claim 4, wherein the EMF shield is positioned between the inner housing portion and inner housing portion.

6. The photobiomodulation system of claim 5, wherein the EMF shield separates light emitting portions of the LED emitters from the operating circuit.

7. The photobiomodulation system of claim 2, wherein the beneficial or harmful EMF shield is a metal foil, coil plate, mesh, a metalized layer on a surface of the housing a substrate, or a metal/polymer composite material.

8. The photobiomodulation system of claim 1, wherein the housing is formed from a metal or composite material configured to shield the patient wearing the housing from the harmful EMF radiation.

9. The photobiomodulation system of claim 1, wherein the LED emitters are configured to emit near infrared light at a wavelength between 650 and 1150 nm.

10. The photobiomodulation system of claim 1, wherein the LED emitters are configured to emit near infrared light at a power density of 25-325 mW/cm² for duration of treatment.

11. The photobiomodulation system of claim 1, wherein the LED emitters are configured to emit near infrared light at a power density of 25-325 mW/cm² for duration of treatment.

12. The photobiomodulation system of claim 9, wherein the LED emitters are configured to emit the near infrared light at frontal lobe locations corresponding to FP1, FP2, Fz, F3, F4, F7, F8 EEG positions.

13. The photobiomodulation system of claim 9, wherein the LED emitters are configured to emit the near infrared light only at frontal lobe locations corresponding to FP1, FP2, Fz, F3, F4, F7, F8 EEG positions.

14. The photobiomodulation of claim 1, wherein the operating circuit includes a wireless communication device, and wherein the wireless communication device causes at least some of the harmful EMF radiation emitted during operation of the LED emitter.

15. The photobiomodulation of claim 1, the housing is configured to shield the patient wearing the housing from 95-99.9% of the harmful EMF radiation.
